(19) United States
(12) Patent Application Publication
(10) Pub. No.: US 2009/0069874 A1
(43) Pub. Date: Mar. 12, 2009

(54) Apparatus for Treating Migraine

(76) Inventor: Saija Aikoma, Helsinki (FI)

Correspondence Address: BIRCH STEWART KOLASCH & BIRCH, PO BOX 747, FALLS CHURCH, VA, 22040-0747, US

(21) Appl. No.: 11/918,602

(22) PCT Filed: Apr. 13, 2006
(86) PCT No.: PCT/FI2006/000114

§ 371 Date: Oct. 16, 2007

(30) Foreign Application Priority Data
Apr. 26, 2005 (FI) U 2005 0147

(51) Int. Cl. A61F 7/00

(52) U.S. Cl. 607/104

(57) ABSTRACT

An apparatus (8) for treating migraine, which apparatus has a two-legged water pipe (3) for distributing water to two or more exit points (2, 2′) for water, and that the apparatus is fastened to a water tap or a water fitting with fastening members 1 and 6, and that the apparatus includes one or more hand-pressable pressing pieces (4) manufactured of flexible material.

This invention relates to an apparatus for treating migraine.

Migraine, that is, an extremely severe headache is a highly common and wide-spread disease. Also nausea and sensitivity to light are related to migraine during a migraine attack.

Medically, migraine is considered to be caused by certain disorders in the functioning of the brain stem, that is, by sensitisation occurring in a certain area of the brain. During a migraine attack and also after it, a patient is frequently incapable of working for a few days, and thus migraine has considerable economic effects as absences from work.

Around 10% of population is calculated to be migraine patients, that is, in Finland, there is approximately 500,000 persons suffering from migraine.

Today, migraine is almost exclusively treated with medicines, and frequently a patient has to use several medicine doses to alleviate his/her migraine condition. When using modern medicines, the medical treatment has, depending on the medicine and the patient, different undesired side effects, and medicines used in the treatment of migraine are additionally very expensive. Furthermore, certain migraine medicines disagree with some migraine patients, because of the contradictions of the medicines, such as, for example, hypersensitivity and interactions with other medicines.

The object of this invention is to minimise and/or eliminate aforementioned disadvantages caused by medicines used in the treatment of migraine and thus to present an alternative solution for use in treating migraine.

In practice, it has been observed that, when a migraine attack is starting and, on the other hand, also when migraine has advanced further, one is able to stop the continuation of the migraine attack already in the beginning of the attack and frequently the advanced migraine is totally even stopped by placing both wrists, or to be precise, the inner parts of wrists under a water spray as warm as possible, most preferably hot and dot-shaped. The water spray should be such that it has a somewhat massaging, that is, pressing effect in certain and desired areas of the wrist. This is provided with a dot-shaped water spray. The temperature of water coming from the water spray should be as hot as possible, however depending on the tolerance of the patient, for achieving an effect as powerful as possible. Simultaneously, as one keeps his/her wrists under the water spray, he/she holds in his/her hands (or possibly only in one hand) a piece manufactured of flexible material which one presses with a pumping motion during the whole treatment. The pumping motion made by hands strengthens the relaxing effect of hot water, improves blood circulation and assists and speeds up the ending of the migraine condition or migraine attack.

A problem of known solutions for distributing water at different points is the slowness of installation and the unsuitability for different taps and water fittings, because there are several different designs of water taps and water fittings on the market.

It is characteristic for a device according to the invention that the apparatus has a two-legged water pipe fastenable with fastening members to a water tap or a water fitting for distributing water to two water exit points, and that the apparatus has one or more hand-pressable pressing pieces of flexible material.

The device according to the invention is further characterised by what is stated in the enclosed patent claims 2-6.

The most important advantage of the invention is that one is able to treat migraine conditions/attacks with the apparatus without medicines and, because of this, the side effects, which may vary depending on a patient, caused by expensive medical treatment, may be totally avoided.

Furthermore, the device according to the invention is safe and easy to use, and it is suitable for installing to most already known water taps and water fittings on the market. The device according to the invention is durable in its structure.

The invention will now be described in detail with reference to the accompanying drawing 1.

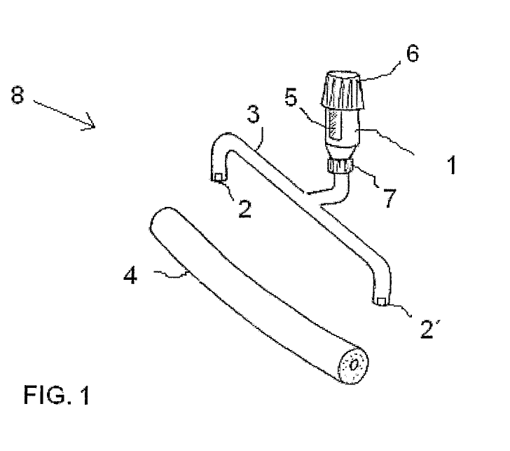

According to FIG. 1, an apparatus 8 comprises a two-legged water pipe 3 the ends of which are bent to an angle of around 90 degrees. In the bent ends, there are exit points 2 and 2′ for water. To one leg of the water pipe is fastened a connector piece 7 in which is located a fastening member 1. The fastening member 1 has a slot 5 in which the end of a water tap or a water fitting is placed and, after this, the end of the water tap or the water fitting is tightened to its place by means of a fastening piece 6 which is plug-shaped and has threads.

In the solution according to the invention, the water pipe may be of flexible material, such as, for example, of copper pipe or aluminium pipe or the like.

The diameter of the exit points for water may be adjustable, whereby it is possible to change the force and the dot-shape of exiting water spray. However, the force of the water spray is adjusted mainly in the water tap or the water fitting into which the apparatus 8 is fastened. Then, one is simultaneously able to adjust the force of both exit points for water to be the same with one adjustment.

A pressing piece 4 pertaining in the apparatus 8, manufactured of flexible material, may be separate from the water pipe, or the pressing piece may be fastened with a string, a tape, a metal wire or some other connecting piece to the water pipe 3.

The design of the pressing piece 4 is either rod- or round-shaped, and it may be manufactured of rubber or plastic or other corresponding flexible, buckling material.

It is well known by those skilled in the art that the different embodiments of the invention are not solely limited to the examples described above, and thus they may vary within the scope of the claims to be presented later.

1. An apparatus (8) for treating migraine, characterised in that the apparatus has a two-legged water pipe (3) fastenable with fastening members (1, 6) to a water tap or a water fitting for distributing water to two exit points (2, 2′) for water, and that the apparatus has one or more hand-pressable pressing pieces (4) manufactured of flexible material.

2. An apparatus according to claim 1, characterised in that the pressing piece has a string, a tape, a metal wire or some other connecting piece for fastening the pressing piece to the water pipe.

3. An apparatus according to claim 1, characterised in that the pressing piece is rod-shaped.

4. An apparatus according to claim 1, characterised in that the pressing piece is round-shaped.

5. An apparatus according to claim 1, characterised in that the pressing piece is manufactured of rubber or plastic or some corresponding flexible material.

6. An apparatus according to claim 1, characterised in that the diameter of the exit points (2, 2′) for water is adjustable.
